(19) United States
(12) Patent Application Publication
(10) Pub. No.: US 2020/0405421 A1
(43) Pub. Date: Dec. 31, 2020

(54) CONTACTLESS DATA AND POWER TRANSMISSION FOR SURGICAL ROBOTIC SYSTEM

(71) Applicant: TransEnterix Surgical, Inc., Morrisville, NC (US)

(72) Inventor: Randall L. Luck, Apex, NC (US)

(21) Appl. No.: 16/051,466

(22) Filed: Jul. 31, 2018

Related U.S. Application Data
(60) Provisional application No. 62/539,531, filed on Jul. 31, 2017.

(51) Int. Cl. A61B 34/00; A61B 34/35; B25J 9/02; A61B 46/20

(52) U.S. Cl. CPC A61B 34/70; A61B 46/20; B25J 9/02; A61B 34/35

(57) ABSTRACT

A system for wireless transfer of power from a robotic arm to a surgical instrument assembly of a surgical robotic system includes a wireless power transmission coil on a robotic arm. A surgical instrument assembly is removably attachable to the robotic arm and includes a surgical instrument shaft and end effector, and a motor operable to actuate or articulate the end effector of the surgical instrument. A wireless power receive coil on the surgical instrument assembly is in electrical communication with the motor.

This application claims the benefit of U.S. Provisional Application No. 62/539,531, filed Jul. 31, 2017, which is incorporated herein by reference.

### BACKGROUND

There are various types of surgical robotic systems on the market or under development. Some surgical robotic systems use a plurality of robotic arms. Each arm carries a surgical instrument, or the camera used to capture images from within the body for display on a monitor. See U.S. Pat. No. 9,358,682 and US 20160058513, which are incorporated herein by reference. Other surgical robotic systems use a single arm that carries a plurality of instruments and a camera that extend into the body via a single incision. See WO 2016/057989, which is incorporated herein by reference. Each of these types of robotic systems uses motors to position and/or orient the camera and instruments and to, where applicable, actuate the instruments, all in accordance with user input. Typical configurations allow two or three instruments and the camera to be supported and manipulated by the system. Input to the system is generated based on input from a surgeon positioned at a master console, typically using input devices such as input handles and a foot pedal. Motion and actuation of the surgical instruments and the camera is controlled by the robotic system controller based on the user input. The image captured by the camera is shown on a display at the surgeon console. The console may be located patient-side, within the sterile field, or outside of the sterile field.

FIG. 1 shows components of a robotic surgical system 10 of the type described in U.S. Pat. No. 9,358,682 and US 20160058513. Features of the system 10 are shown to facilitate an understanding of the way in which the concepts of the present invention may be implemented, but it should be understood that the invention may be used with a variety of different surgical or industrial robotic systems and is not limited to use with system 10.

System 10 comprises at least one robotically controlled arm 11 which operates under the control of a command console 12 operated by the surgeon, as described above. The system described in U.S. Pat. No. 9,358,682 includes a manipulator wrist as part of the distal end of the robotic arm 11. The robotic arm has a distal portion or terminal portion 13 (e.g. at the manipulator wrist in embodiments having such a design) designed to support and operate a surgical device assembly 14. The surgical device assembly includes a surgical instrument having shaft 15 and a distal end effector 17 positionable within a patient 16. The robotic arm is moveable by the system (e.g. in response to user input at the console) to position and orient the surgical instrument within the patient 16.

In this configuration, the robotic arm 11 receives the surgical device assembly 14 at the terminal portion 13 as shown in FIG. 2. The surgical device assembly includes a proximal housing 20 that is received by the terminal portion 13 as shown.

The end effector 17 may be one of many different types of that are used in surgery including, without limitation, end effectors 17 having one or more of the following features: jaws that open and close, a section at the distal end of the shaft that bends or articulates in one or more degrees of freedom, a tip that rolls axially relative to the shaft 15, a shaft that rolls axially relative to the robotic arm 11. For the sake of simplicity, in FIG. 2 the end effector 17 is shown as an oval form in broken lines.

The system includes instrument actuators for driving the motion of the end effector 17. These actuators, which might be motors or other types of motors (e.g. hydraulic/pneumatic), are positioned in the terminal portion 13 of the robotic manipulator, or in the housing 20 of the surgical device assembly, or some combination of the two. In the latter example, some motion of the end effector might be driven using one or more motors in the terminal portion 13, while other motion might be driven using motors in the housing 20.

The robotic arm 11 is typically provided non-sterile. During surgery, it is covered with a sterile drape or barrier 18a as shown in FIG. 2. The surgical instrument (shaft and end effector 15, 17) is provided as a sterile component, and in some cases the housing 20 of the surgical device assembly is also a sterile component and can be mounted directly onto the sterile barrier 18a. In other cases, the housing contains motors or sensitive electronics and thus cannot be subjected to sterilization processes. In those cases, a second sterile barrier 18b such as a sterile bag is positioned around the housing before it is mounted onto the robotic arm. In the configuration described in US 20160058513, once the housing 20 is mounted onto the robotic arm, conductive pins in the housing or the arm are caused to pierce the sterile barriers, creating an electrical connection between components of the arm and electronic components, electromechanical actuators, and/or sensors of the housing 20. This connection allows communication of power used to power the motors within the housing 20.

It is desirable to provide a robotic surgical system that allows power and control data to be provided to a surgical instrument assembly without making any electrical contact. Additionally feedback of data from the surgical instrument assembly back to the main system is also desirable and is described. Using the embodiments disclosed herein, power transfer and bidirectional data transfer can be achieved without puncturing the surgical drape covering the robotic arm.

### BRIEF DESCRIPTION OF THE DRAWINGS

FIG. 1 schematically illustrates a robotic manipulator of a type used in robotic surgical procedures.

FIG. 2 illustrates the step of mounting of a surgical device onto the manipulator of FIG. 1.

FIG. 3 illustrates a distal part of a robotic surgical arm and a surgical instrument assembly mountable on the arm together with a system for wireless and contactless power and data transfer in accordance with principles described herein.

FIG. 4 is a schematic diagram of a system for wireless and contactless power and data transfer in accordance with principles described herein.

FIG. 5 is a schematic diagram of a 50 watt power transmitter link prototype

FIG. 6 is a schematic diagram of a 50 watt power receiver link prototype

FIG. 7 is a schematic of a 13.56 MHz data transmitter link prototype; and

FIG. 8 is a schematic of a 13.56 MHz data receiver link prototype.

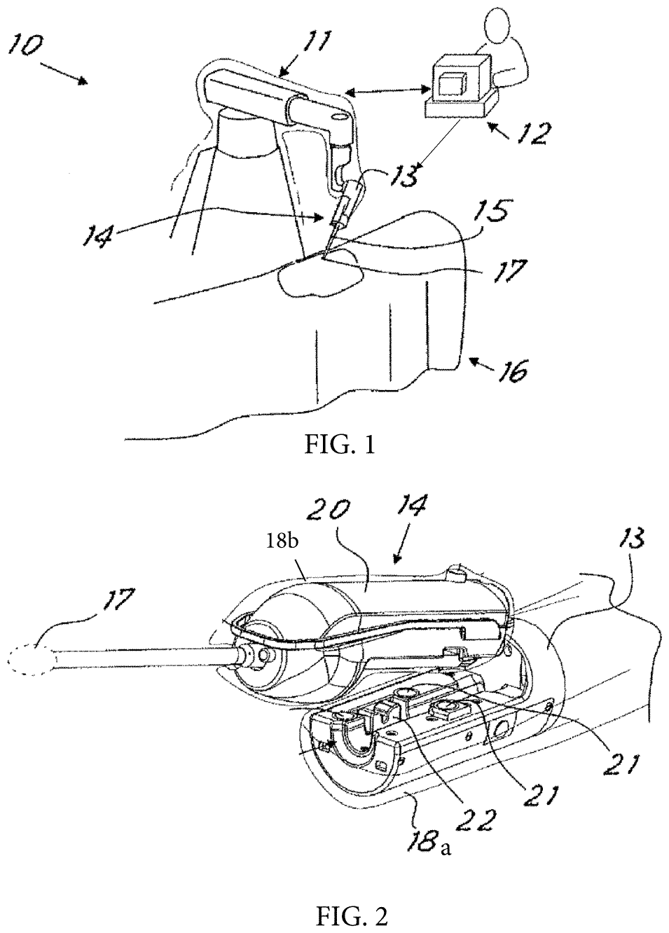

### DETAILED DESCRIPTION

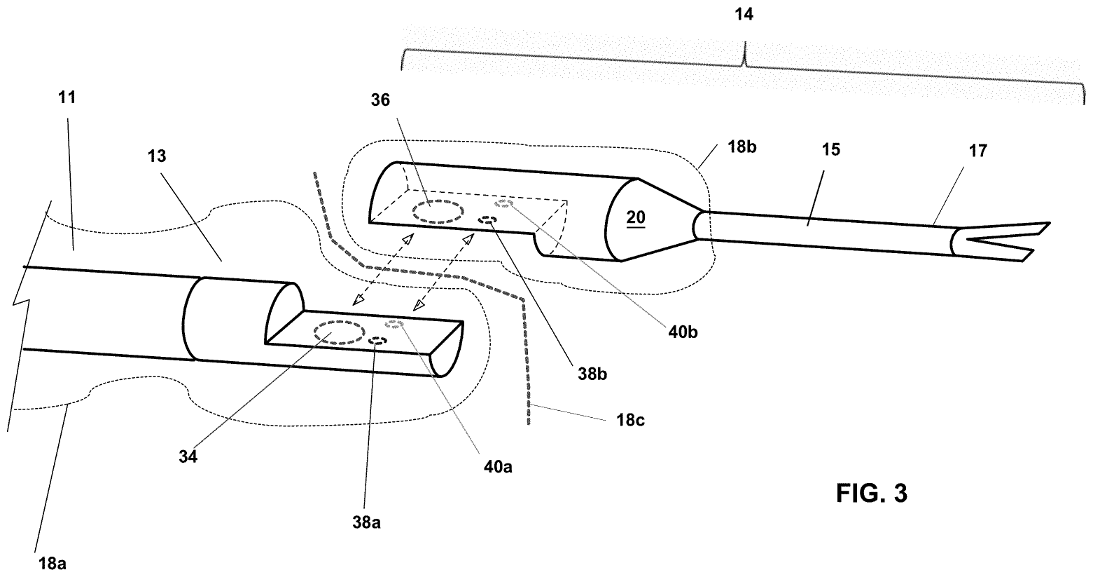

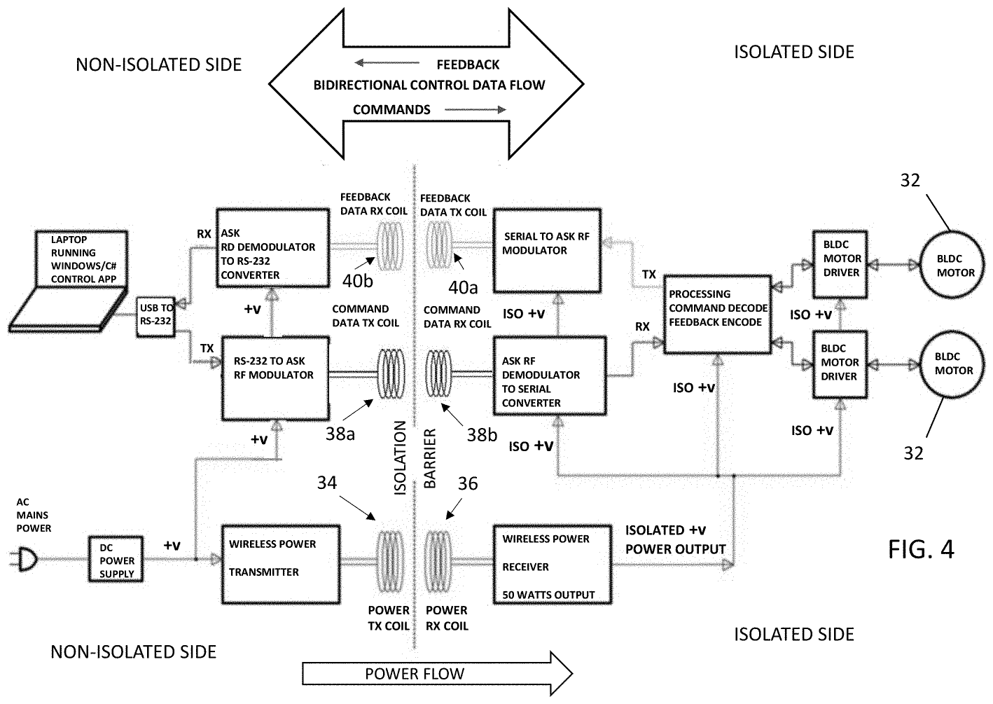

There are two concepts which taken together form the preferred form of this invention. The first is wirelessly transmitting enough power to run motor driven end effectors and other instruments. The second is wirelessly transmitting control and data information in a full duplex bidirectional manner. FIG. 3 shows these concepts incorporated into a robotic surgical configuration of the type shown in FIG. 2. A schematic diagram of the wireless and contactless power and data transfer system is shown in FIG. 4. As shown in FIG. 4, the system allows (a) power and control data to be provided to components of the surgical instrument assembly and (b) feedback of data from the motors and electronics of the surgical instrument assembly back, each without the need for electrical contact.

The disclosed system may be incorporated into a system having the features described in the background section. Turning now to FIG. 3, in one embodiment, the system is incorporated into a robotic arm 11 configured to removably receive a surgical device assembly 14 comprising a shaft 15 and an end effector 17. FIG. 3 shows the surgical device assembly 14 separated from the robotic arm 11 to allow the two components to be more easily seen.

One or more sterile barriers are positionable between the robotic arm 11 and the surgical device assembly 14 during use. In FIG. 3, a first sterile barrier 30a covers at least the distal portion 13 of the robotic arm 11, and a second sterile barrier 30b covers a proximal portion of the surgical device assembly 14 such as the housing 20 and a proximal section of the surgical instrument. FIG. 3 also shows a third sterile barrier 30c placed between the distal portion 13 of the robotic arm and the surgical device assembly. The third sterile barrier is positioned such that when the surgical device assembly is mounted to the robotic arm the third sterile barrier 30c is sandwiched between them.

As described in the background section, the end effector 17 may be one of many different types that are used in surgery including, without limitation, those having one or more of the following features: jaws that open and close, a section at the distal end of the shaft that bends or articulates in one or more degrees of freedom, a tip that rolls axially relative to the shaft 15, a shaft that rolls axially relative to the manipulator robotic arm 11. The end effectors might additionally be equipped to deliver energy to tissue for cutting, coagulation, sealing or for some other therapeutic or diagnostic purpose. In other embodiments, the surgical device assembly 14 may be a camera, laparoscopic camera, or endoscope in which case the surgical device may or may not be one that bends or articulates. In other embodiment, the surgical device might also be an illuminator, an OCT probe, a fiber-based spectrometer, an optical or RF tissue-treatment device, an optical tissue interrogator, or an ultrasound probe

Where the surgical device includes an actuatable end effector, the system includes one or more instrument actuators 32 for driving at least some of the motions or actions of the end effector 17. These actuators are preferably motors although other types of actuators could be used. Some or all of the actuators 32 are positioned in the housing 20 of the surgical device assembly. As noted in the background section, in other embodiments there may be some of the actuators 32 in the housing while others are within the robotic arm 11. As a non-limiting example of this alternate embodiment, actuators in the robotic arm might drive some movement(s) or function(s) of the end effector (e.g. jaw open/close or one of the other functions listed above) by mechanically transferring motion to the surgical device assembly 14 from one side of the sterile barrier(s) to the other side (see, for example, US 20160058513), while actuators 32 in the housing 20 might drive one or more different ones of the functions listed above, such as end effector articulation, bending, tip roll etc. If the surgical device is a camera or other instrument that is not designed for bending or articulation or other end effector motion, the actuators 32 may be omitted from the relevant robotic arm or surgical device assembly 20 but the power transfer features described here may be used to power other functions of those instruments.

The robotic arm 11 includes a wireless power transmit coil 34. The wireless power transmit coil is in wireless communication with a wireless power receive coil 36 in the surgical device assembly 14. The wireless power receive coil 36 may be in the housing 20 or some other part of the surgical device assembly. The arrangement of coils 34, 36 allows wireless transmission of power to run the actuator(s), camera components, and other components of the surgical device assembly that require power. This arrangement will be discussed in further detail in the section below entitled “Power Transfer.”

The robotic arm 11 also includes a wireless data transmit coil 38a that is in wireless communication with a wireless data receive coil 38b carried by the surgical device assembly 20, and a wireless data receive coil 40b that is in wireless communication with a wireless data transmit coil 40a carried by the surgical device assembly 20. This system of wireless data transmission and receiving coils enables simultaneous bi-directional communication between the surgical device assembly 14 and the robotic system. In other words, data can flow from the surgical device assembly to the robotic arm simultaneously with the flow of data from the robotic arm to the surgical device assembly. More details are described below in the section entitled “Bi-directional Transmission/Receipt of Data.”

Many types of data may be communicated between the instrument and the system and this invention is not intended to be limited to communication of specific types of data. Examples of the types of information that may be communicated between the surgical device assembly and the robotic arm (and thus the robotic system) include commands to components of the surgical device assembly (including the actuators 32), feedback from sensors and other components of the surgical device assembly including the motors and associated components, and information stored on or collected from components of the surgical device assembly. Feedback may include data relating to information sensed or detected by sensors on the surgical device assembly. This may include data from force sensors or motor encoders, the feedback from which can be used by the robotic system to determine determining grasping forces or other tissue contact forces and generate haptic feedback that is delivered to the user at the surgeon console (e.g. at the control handles of the surgeon console), or diagnostic information etc. Information transmitted from the surgical device to the robotic system might also be of the type that identifies properties of the surgical instrument (e.g. the type of instrument, its dimensions or other physical properties) so that the robotic system controller can properly control motion and actuation of the surgical device in use. The information transmitted might additionally or alternatively include usage information such as the number of times the instrument has been used, the number of “uses” remaining before the instrument must be discarded, the amount of time the surgical device has been used, or instructions to update the usage information stored on the surgical device. Image information and/or camera control information may also be transferred if the surgical device is a camera.

Power Transfer

The configuration for wirelessly transmitting power is configured to transfer enough power to run motors in the surgical instrument assembly or motor pack 20 in order to drive motion or actuation of the end effectors (e.g. jaw open/close, articulation or bending in one or more degrees of freedom, instrument tip roll etc), and/or to operate other components of the surgical device such as lights, camera features, etc.

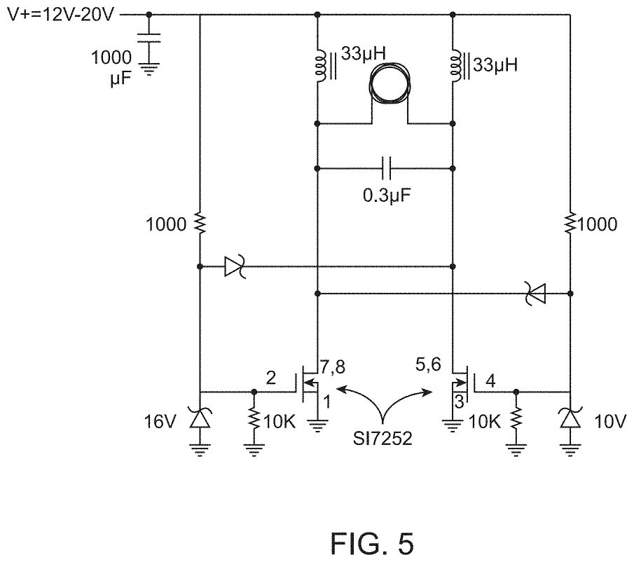

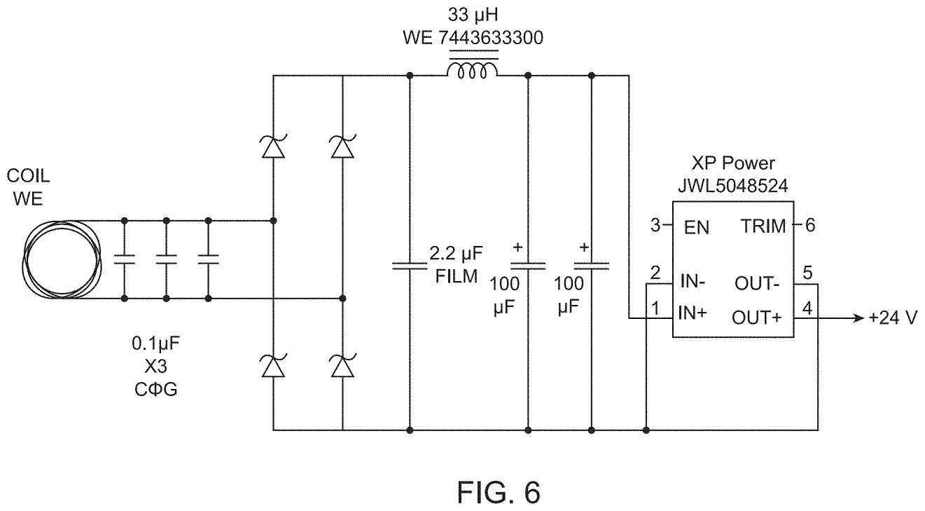

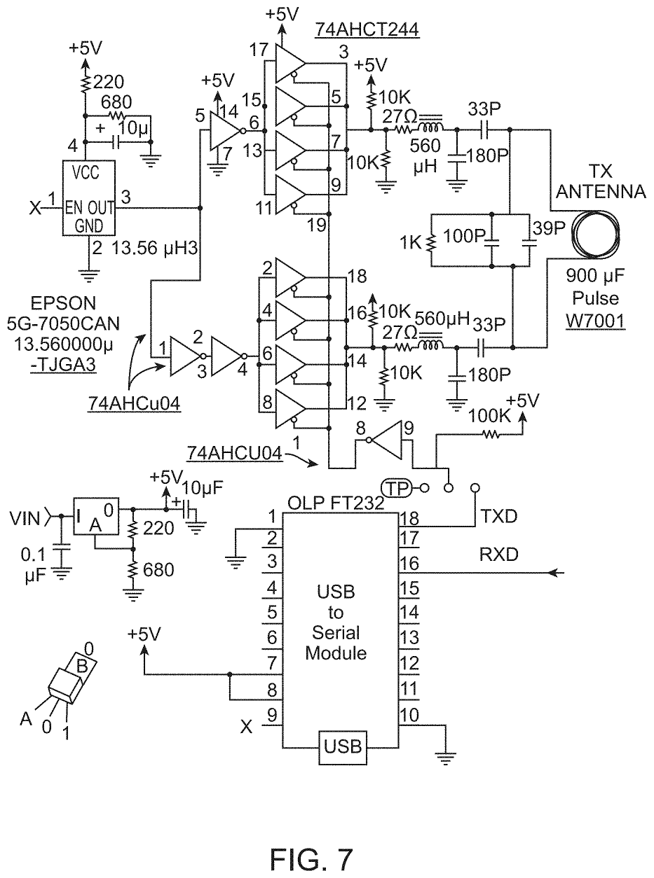

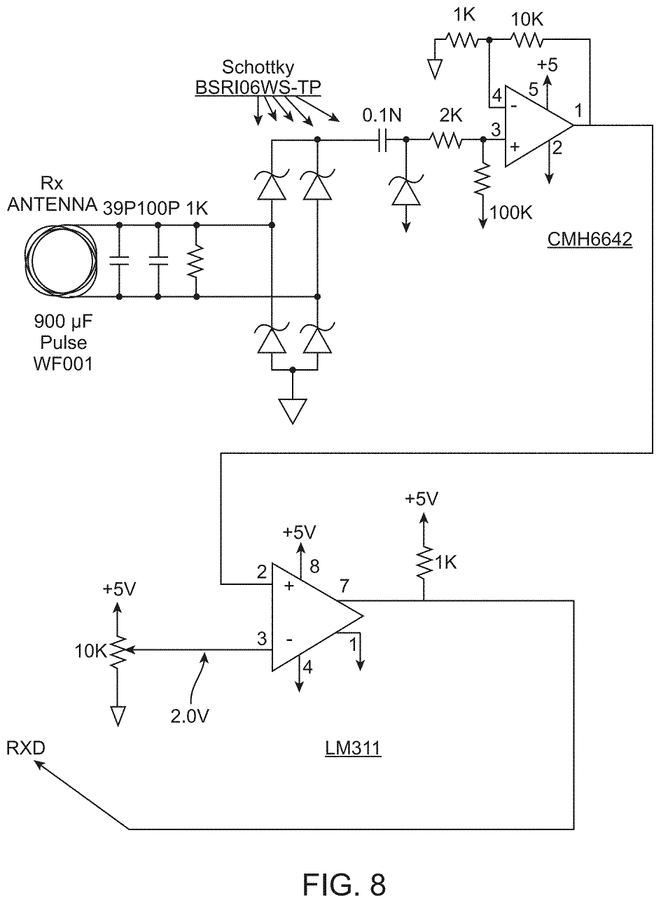

The method of transferring power uses two flat coil inductors coils that are separated by insulating barriers and positioned as discussed with respect to FIG. 3 These coils are generally commercially available for use in contactless cell phone chargers that use the “Qi” standard. These coils have a ferrite material on one side so that they can be placed on a metal surface. FIGS. 5 and 6 show schematics of prototype transmitter and receiver prototypes, respectively. The transmitter coil is connected in parallel with a capacitor such that the combination is electrically resonant somewhere in the range of 100 kHz to 200 kHz. This particular frequency band is useful because significant power can be radiated while still staying below the allowed FCC radiation limits. The formula for finding the frequency of resonance is f=1/(2×pi×sqrt(L×C)). So for example, if L=6.3 uH and C=0.3 uF, then the resonant frequency is about f=115.8 kHz. The power coil can be driven in several different ways. One way is to use an H-bridge type transistor or MOSFET driver circuit. Another way is to use the resonant LC circuit as a tank for a power oscillator. The first way is more controlled but it requires that the frequency that drives the H-bridge is controlled by some other means to keep the LC circuit in resonance. Additionally, at high powers, the H-bridge drive can create harmonics that might be hard to keep from radiating out as interference to other devices. The Qi cellphone charging standard works in this way. It uses a back channel control link to adjust the master oscillator frequency that drives the H bridge so that it is kept at the resonant frequency. In contrast, the second way automatically by virtue of its design, always oscillates at the resonant frequency of the tank circuit. Additionally, it oscillates as a clean sine wave which means that there are little to no harmonics to radiate. When a second coil with a second parallel capacitor is physically placed parallel and close to the transmitter coil, the two coils become resonant together. They become an inductively coupled near field link. The oscillator circuit on the transmit side naturally changes frequency a bit when the second coil is placed nearby (a cm or so).

Significant power can be transmitted in this configuration. In the prototype system, 50 watts of power were transferred with a 20V DC input. See also Wurth Electronics application note ANP032e, incorporated herein by reference, for further details about this type of tank circuit power oscillator.

### Bi-Directional Transmission/Receipt of Data

A first method of bi-directionally transmitting and receiving data also uses inductively resonant near field coupled coils. The inventor of the described concepts has built in demo circuits a system that uses a carrier frequency of about 80.5 kHz. The coils are 13 uH and about a cm in diameter. The signal that drives the transmitter coil is modulated on and off with the 1's and 0's of the serial data stream thus creating an amplitude modulated system. Due to the low carrier frequency, this system only allows data rates up to about 600 baud. The receiver coil then passes the amplitude modulated signal to a full wave bridge and a simple AM detector. A second demo system uses the 13.56 MHz ISM band that most HF RFID (radio frequency identification) and NFC (near field communications) utilizes. All worldwide radio standard agencies allow a narrow band around this 13.56 MHz frequency to be used license free with no power limits. This particular frequency is in what is called one of the ISM bands (industrial, scientific, medical). In the demo system the coils are 0.9 uH and about 2 cm square. Since this communications link is in the near field, the RF radiation is physically limited and confined to the vicinity of the coils. The radiated power drops off very quickly, proportional to 1 over the 6^{th} power of distance. In alternative embodiment, one might use other non-ISM frequencies in a manner that keeps the stray radiated power stays within acceptable limits set by the standards. In order for the data link to be bidirectional, each side has a separate transmitter coil and a receiver coil. Additionally, each side has separate transmitter and receiver circuits. This allows simultaneous transmission in both directions, also called full duplex communication. The data rate possible is as much as 1 M baud.

In an alternative embodiment, the existing technology of NFC (near field communications) could be used as the data path link, however NFC lacks the true full duplex bidirectional communications link capabilities of the configuration described above. Instead, it is half duplex where one side transmits and the other side has to wait before transmitting. NFC systems and HF RFID systems have only one transmitter. The receiver sends data back to the transmitter side by selectively loading its resonant coil circuit with the pattern of the data. The transmitter then has a means to sense this slight load variation and can demodulate this return channel data. (This is called “backscatter” in RFID and NFC terminology.) However, this configuration is unable to transmit data while it is receiving and vice versa.

Other systems may use a technology like Bluetooth (at 2.54 GHz) for the data transmission link. This however is not a near field data transmission system, rather is a true far field wireless radio link, and possibly subject to greater interference, jamming, or even eavesdropping.

The system described in this application has numerous advantages, including the ability to provide contactless, wireless power and data communications from a surgical robotic arm to components of a completely surgical device assembly housing thus preserving the sterile barrier which is important in the operating room. It does so while providing sufficient transmitted power to run the end effector's motors. For data communication, it gives the added advantage of using two sets of coils, one set for each data direction so that the data communications is full duplex. This will be useful in the context of providing haptic feedback to the user based on forces sensed by sensors of the surgical device assembly so that the surgical device assembly can provide continuous tactile feedback to the user or the controller system with minimal latency.

It should be understood that while the preferred form of this invention combines wireless transmission of power with wireless transmission of control and data information in a full duplex bidirectional manner, it should be understood that the system may be modified in certain ways to exclude some features without departing from the scope of the invention. For example, one alternative embodiment might make use only the power transmission features described above. Another might make use only of the bi-directional data transmission. Still others might combine the power transmission aspect with un-directional transmission of data in one direction or the other, or with other types of data transmission.

1. A system for wireless transfer of power from a robotic arm to a surgical instrument assembly of a surgical robotic system, the system comprising:
a robotic arm including a wireless power transmission coil on the robotic arm;
a surgical instrument assembly removably attachable to the robotic arm, the surgical instrument assembly including a surgical instrument shaft and a motor operable to cause operation of the surgical instrument; and
a wireless power receive coil on the surgical instrument assembly in electrical communication with the motor.

2. The system of claim 1, wherein the operation of the surgical instrument is at least one of jaw open, jaw close, articulation or bending in at least one degree of freedom, and instrument tip or shaft roll.

3. The system of claim 1, including a sterile drape between the robotic arm and the surgical instrument assembly.

4. The system of claim 1, wherein the surgical instrument assembly includes a plurality of motors operative to drive a plurality of operations of the surgical instrument, each motor in electrical communication with the power receive coil.

5. The system of claim 1, wherein the system is further for wireless communication of data between the surgical instrument assembly and the surgical system or robotic arm:
the robotic arm further includes a wireless data transmit coil and a wireless data receive coil;
the surgical instrument further includes a wireless data transmit coil and a wireless data receive coil,
wherein the wireless data transmit coils and wireless data receive coils are operable for simultaneous bidirectional communication of data between electronic components of the surgical instrument assembly and electronic components of the robotic arm or surgical system.

6. A system for wireless communication of data between a robotic arm or robotic surgical system to a surgical instrument assembly of a surgical robotic system, the system comprising:
a robotic arm including a wireless data transmit coil and a wireless data receive coil;
a surgical instrument assembly removably attachable to the robotic arm, the surgical instrument assembly including a surgical instrument shaft and a motor operable to cause operation of the surgical instrument; and
a wireless data transmit coil and a wireless data receive coil on the surgical device assembly,
wherein the wireless data transmit coils and wireless data receive coils are operable for simultaneous bidirectional communication of data between electronic components of the surgical instrument assembly and electronic components of the robotic arm or surgical system.

7. The system of claim 6, including a sterile drape between the robotic arm and the surgical instrument assembly.
